(19) United States
(12) Patent Application Publication
(10) Pub. No.: US 2023/0149546 A1
(43) Pub. Date: May 18, 2023

(54) PHOTOSENSITIZER CONJUGATED GOLD NANOPARTICLES FOR RADIOTHERAPY ENHANCEMENT

(71) Applicant: BioVentures, LLC, Little Rock, AR (US)

(72) Inventors: Samir Jenkins, Little Rock, AR (US); Robert Griffin, Little Rock, AR (US)

(21) Appl. No.: 17/919,454

(22) PCT Filed: Apr. 16, 2021
(86) PCT No.: PCT/US2021/027692

§ 371 Date: Oct. 17, 2022

Related U.S. Application Data
(60) Provisional application No. 63/011,745, filed on Apr. 17, 2020.

(51) Int. Cl. A61K 41/00; A61K 47/69; A61K 47/60; A61P 35/00; A61N 5/06

(52) U.S. Cl. CPC A61K 41/0071; A61K 47/6929; A61K 47/6923; A61K 47/60; A61P 35/00; A61N 5/062; A61N 2005/0663

(57) ABSTRACT

Among the various aspects of the present disclosure is the provision of compositions and methods for targeted treatment of cancers or neoplasms. In particular, the present disclosure is directed to a photodynamic system comprising a nanoparticle conjugated to a photosensitizer that can be activated by an excitation source such as ionizing radiation or other forms of electromagnetic energy.

### CROSS REFERENCE TO RELATED APPLICATIONS

This application claims the benefit of U.S. Provisional Application No. 63/011,745, filed Apr. 17, 2020 the disclosure of which is herein incorporated by reference in its entirety.

### FIELD OF THE TECHNOLOGY

The disclosed subject matter relates generally to cancer therapy, more particularly to compositions and methods for treating cancer by photodynamic activation of photosensitizers in a tissue affected by a cancerous condition.

### BACKGROUND

Photodynamic Therapy (PDT) is a therapeutic procedure to destroy tissue, preferably pathological tissue, for example, cancer tissue or tissue in blood vessels that occur in disorders characterized by hypervascularization or proliferation of neovascular networks. In cancer, PDT can be locally administered as a primary therapy for early stage disease, palliation of late stage disease, or as a surgical adjuvant for tumors that show loco-regional spread.

In PDT, a photosensitizing agent (termed a “photosensitizes”) is delivered to the target tissue and then radiation, most usually light of wavelengths between 250-1000 nm is applied to the target tissue. Thus, photosensitizing agents are activated by electromagnetic (EM) radiation. This activation results in the photochemical transfer of the energy by the photosensitizer-molecules to a variety of other molecules in the tissue, resulting in the generation of reactive radical species including, amongst others, singlet oxygen, the superoxide radical, and peroxides and peroxide radicals. The activation of the photosensitizing agent in the tissue leads to, amongst other processes, the generation of radicals and, ultimately, the destruction of the target tissue, or the initiation of biological processes that result in the desired effect upon the target tissue.

However, the limited penetrability of light in tissues remains a large limiting factor in the use of PDT for the treatment of cancer, specifically cancers located within deeper tissue. Therefore, there is a need in the art exists for photodynamic therapy systems and methods for treating diseases such as tumors, located deep under the skin, a result of short penetration depth of light in tissues. Furthermore, there is a need to reduce off target toxicities.

### SUMMARY

The present disclosure is based on the provision of compositions for targeted treatment of cancers or tumors and to methods of making and using the same.

One aspect of the present disclosure provides a photodynamic therapy system which includes a nanoparticle comprising a photosensitizer conjugated thereto, wherein excitation of the nanoparticle results in excitation of the photosensitizer and the nanoparticle is present in an effective size and amount to activate the photosensitizer upon absorption of the excitation source. In some embodiments, the nanoparticle is a gold nanoparticle. In some embodiments, the photosensitizer has a zero-length conjugation to the nanoparticle. In some embodiments, the photosensitizer is covalently attached to the surface of the nanoparticle through a polyethylene glycol moiety.

In one aspect of the disclosure, the photosensitizer is derived from cyanine, porphyrin and their derivatives, pyrrole, tetrapyrollic compound, expanded pyrrolic macrocycle and their derivatives, flavins, organometallic specie, or combinations thereof. In some embodiments, the photosensitizer is derived from cyanine selected from the group consisting of merocyanine, phthalocyanine, chloroaluminum phthalocyanine, sulfonated aluminum phthalocyanine, ring-substituted cationic phthalocyanine, disulfonated or tetrasulfonated derivative, sulfonated aluminum naphthalocyanine, naphthalocyanine, tetracyanoethylene adduct, crystal violet, azure β chloride, benzophenothiazinium, benzophenothiazinium chloride (EtNBS), phenothiazine derivative, rose Bengal, toluidine blue derviatives, toluidine blue O (TBO), methylene blue (MB), new methylene blue N (NMMB), new methylene blue BB, new methylene blue FR, 1,9-dimethylmethylene blue chloride (DMMB), methylene blue derivatives, methylene green, methylene violet Bernthsen, methylene violet 3RAX, Nile blue, Nile blue derivatives, malachite green, Azure blue A, Azure blue B, Azure blue C, safranine O, neutral red, 5-ethylamino-9-diethylaminobenzo[a]phenothiazinium chloride, 5-ethylamino-9-diethylaminobenzo[a]phenoselenazinium chloride, thiopyronine, and thionine.

In some embodiments, the photodynamic therapy system uses an excitation source which produces radiation selected from the group consisting of X-rays, alpha particles, beta-particles, neutrons, gamma rays, and combinations thereof. In other aspects, the nanoparticle upon excitation by the excitation source is characterized as a scintillation nanoparticle. In still another embodiment, the system comprises a cell recognition moiety such as a receptor, ligand, polynucleotide, peptide, polynucleotide binding agent, antigen, antibody, or combinations thereof.

In another aspect, the disclosure provides a method for photodynamic therapy in a subject by (1) providing a photodynamic system including a nanoparticle comprising a photosensitizer conjugated thereto, wherein excitation of the nanoparticle results in excitation of the photosensitizer and the nanoparticle is present in an effective size and amount to activate the photosensitizer upon absorption of the excitation source; and (2) providing an excitation source, wherein the excitation source is capable of exciting the nanoparticle and thereby exciting the photosensitizer to provide the photodynamic therapy and also combining with the possible therapeutic effects of the excitation source to generate an additive or synergistic result.

Other features or advantages of the present invention will be apparent from the following drawings and detailed description of several examples, and also from the appended claims.

### BRIEF DESCRIPTION OF THE FIGURES

FIG. 1 shows extinction spectra for gold nanoparticles without (solid) and with (dashed) Ce6 conjugation.

FIG. 2 shows reactive oxygen species generation relative to untreated groups. Cells were incubated with particles overnight and irradiated with 10 Gy.

FIG. 3 shows clonogenic survival of cancer cells following overnight incubation with the listed material.

FIG. 4 shows the percentage of necrotic tissue in harvested tumors following H&E staining 10 days after irradiation.

FIG. 5 shows tumor growth following intratumoral injection of listed material and 20 Gy irradiation.

FIG. 6 shows a synthetic scheme of an exemplary nanoparticle conjugate (AuNP-Ce6).

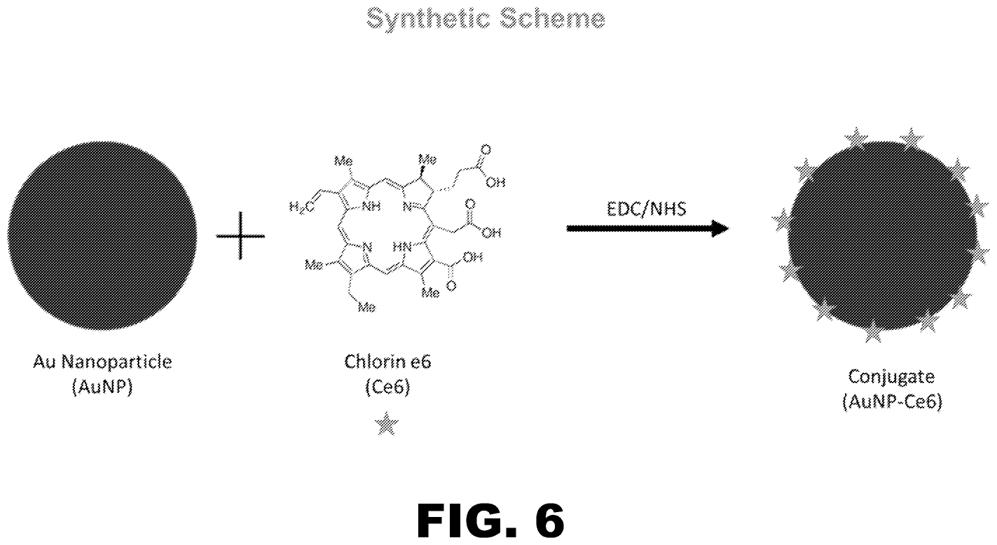

### DETAILED DESCRIPTION

The present disclosure is based, at least in part, on the discovery that the nanoparticle compositions according to the disclosure function to increase radiation-induced cell killing compared to traditional radiation or photodynamic therapy methods. In addition, the nanoparticle compositions according to the disclosure are useful to increase radiation-induced necrosis and cause growth delay of cancerous cells. In some embodiments, the present disclosure provides nanoparticle compositions which function as a nanoparticle delivery system of a photosensitizer resulting in more stability, less toxicity, and targeted delivery of the photosensitizer to tumor or cancer cells. In addition, the nanoparticle compositions according to the present disclosure overcome the limitations of associated with photodynamic therapy, namely, the ability to activate the photosensitizer for photodynamic therapy at deeper tissue depths compared to the short penetration depth of light in tissues.

Cancer is responsible for 25% deaths in the USA, with estimated 1,685,210 new cases and 595,690 people will die from the disease each year. An estimated 14.1 million new cases and 8.2 million deaths from the disease each year worldwide. Despite the fact that the success rate for chemotherapy has risen every year for the past decade by extending the life of patients by reducing the risk of cancer recurrence, it is related to multiple adverse effects. Chemotherapy is a systemic therapy working through the whole body, which kills cancer cells but also healthy cells in the bone marrow, digestive tract, and hair follicles, which may cause life-threatening infections due to leukopenia, loss of appetite and alopecia (hair loss). The emphasis in cancer treatment in general has shifted from cytotoxic, non-specific chemotherapies to molecularly targeted, rationally designed therapies promising greater efficacy.

Most targeted therapies specific for cancer cells are either small molecules (targeting mainly intracellular components), monoclonal antibodies (targeting cell surface molecules), and nanoscale technologies are changing the scientific landscape in terms of cancer detection, treatment, and prevention. The small size, improved solubility, and customized surface by “decorating” NPs thus superior cancer targeting, and multi-functionality by “loading” NPs with therapy provides number of biomedical applications. However, synthetic drug delivery systems have considerable pharmacokinetic and pharmacodynamic disadvantages, such as accumulation in the liver and other filtrating organs. A better strategy to specifically target any cancer cells and improve drug delivery for better treatment efficacy, while reducing side effects in normal tissues are urgently needed.

Photodynamic therapy (PDT) is based on the use of light-sensitive molecules. When light-sensitive molecules are activated by light at specific wavelengths, they cause a variety of active forms of oxygen to be created, the main one of which is singlet oxygen. The process involves absorption of photons by the light-sensitive molecule to produce an excited state which, ultimately, transfers its energy to available surrounding oxygen to produce a molecular excited state of oxygen in the singlet state. This reaction is common to essentially all light-sensitive molecules currently being studied for possible applications in PDT. The formation of singlet oxygen in cell membranes, cytoplasm or organelles results in peroxidative reactions that cause cell damage and death. Administration of the light-sensitive molecule, followed, at the appropriate time, by light treatment using a wavelength that activates the light-sensitive molecule, may result in effective ablation of the targeted tissue. However, PDT is limited to superficial tissues, due to the penetration depth of visible light, and is prone to off target toxicities.

A nanoparticle composition of the disclosure utilizes a nanoparticle capable of absorbing radiation and emitting electromagnetic radiation (e.g., visible light) of a first wavelength and conjugated to the nanoparticle is a photosensitizer that absorbs electromagnetic radiation of said first wavelength. Absorption of the first wavelength by the photosensitizer can activate the photosensitizer to produce singlet oxygen for photodynamic therapy. Accordingly, a nanoparticle composition as disclosed herein provides enhanced radiation-induced therapy that specifically targets tumor or cancer cells and avoids normal tissues. In some embodiments, the disclosed nanoparticle composition may be used for used for therapeutic and/or diagnostic approaches in cancer.

Discussed below are components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular component is disclosed and discussed and a number of modifications that can be made to a number of molecules of the component are discussed, specifically contemplated is each and every combination and permutation of the component and the modifications that are possible unless specifically indicated to the contrary. Thus, if components A, B, and C are disclosed as well as a component D, E, and F and an example of a combination composition, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Various aspects of the invention are described in further detail in the following sections.

### I. Compositions

A composition of the present disclosure may comprise one or more active agents. In some embodiments, an active agent may be an agent to treat, or reduce a neoplasm or cancer. In some embodiments, treating or reducing a neoplasm or cancer may comprise slowing the growth of a neoplasm or cancer cell. In some embodiments, treating or reducing a neoplasm or cancer may include killing a neoplasm or cancer cell. A composition of the disclosure may further comprise a pharmaceutically acceptable excipient, carrier, or diluent. Further, a composition of the disclosure may contain preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents, or antioxidants.

The present disclosure relates to compositions of a nanoparticle composition and methods of using a nanoparticle composition to treat or reduce a cancer or tumor. A nanoparticle composition of the disclosure is useful for photodynamic therapy. The nanoparticle composition can comprise a nanoparticle that emits electromagnetic radiation having a first wavelength, a photosensitizer that absorbs electromagnetic radiation of said first wavelength, wherein the photosensitizer is conjugated to the surface of the nanoparticle. In some examples, excitation of the nanoparticle by electromagnetic radiation having a second wavelength, such as X-rays, can cause the nanoparticles to emit electromagnetic radiation of a first wavelength. Absorption of the first wavelength by the photosensitizer can activate the photosensitizer to produce singlet oxygen for photodynamic therapy.

Other aspects of the disclosure are described in further detail below.

a) Nanoparticle Composition

The present disclosure provides for a nanoparticle composition. In some embodiments, the composition comprise nanoparticles that have at least one photosensitizer conjugated to the surface of the nanoparticles. In some embodiments, the composition comprises nanoparticles having at least one photosensitizer and at least one targeting moiety conjugated to the surface of the nanoparticles. A composition of the present disclosure may also comprise a suitable pharmaceutically acceptable carrier known in the art.

As used herein, the term nanoparticle refers to a particle that has a diameter of at least one region with a dimension (e.g., length, width, diameter, etc.) of less than about 1,000 nm. In some embodiments, the dimension is smaller (e.g., less than about 500 nm, less than about 250 nm, less than about 200 nm, less than about 150 nm, less than about 125 nm, less than about 100 nm, less than about 80 nm, less than about 70 nm, less than about 60 nm, less than about 50 nm, less than about 40 nm, less than about 30 nm or even less than about 20 nm). In some embodiments, the dimension is between about 20 nm and about 250 nm (e.g., about 20, 30, 40, 50, 60, 70, 80, 90, 100, 110, 120, 130, 140, 150, 160, 170, 180, 190, 200, 210, 220, 230, 240, or 250 nm). Nanoparticles may be substantially spherical in shape and the diameter of a group of nanoparticles may be represented by the average diameter of the nanoparticles in the group.

A nanoparticle that can be used according to the present disclosure should be capable of absorbing radiation and emitting electromagnetic radiation (such as visible light) of a first wave length. The nanoparticle can be metallic nanoparticles, organic nanoparticles, hydrolytic nanoparticles, inorganic nanoparticles, ceramic nanoparticles, doped nanoparticles, and combinations thereof. Generally, the nanoparticle is selected such that, the electromagnetic radiation emitted has a wavelength that overlaps, at least partially, with the absorption and/or emission spectrum of the photosensitizer. For example, if the photosensitizer is chlorin e6, the selected nanoparticle can have a maximum emission wavelength of about 650 nm to match the absorption band of chlorin e6.

The nanoparticle can be a scintillation nanoparticle. Scintillation nanoparticles, as used herein, refer to nanoparticles that can absorb ionizing radiation such as X-rays, neutrons, alpha, beta, or gamma-rays. Following irradiation, the nanoparticles become excited and the radiative recombination of electron hole pair results in an afterglow of visible light, that is, a scintillation. In some embodiments, the nanoparticles contain metal atoms capable of absorbing x-rays. Accordingly, a nanoparticle according to the disclosure can be a high-Z metal nanoparticle. High-Z metal NPs are widely used in nanomedicine because of their unique abilities such as photothermal effect, fluorescence for optical imaging, photoacoustic effect, and radiosensitizing effects. Since high-Z metal NPs have a higher stopping power for ionizing radiation than soft tissue, they result in enhanced radiotherapy efficacy. Radiosensitization mechanism by high-Z metal nanoparticles can be explained by two different aspects, physical dose enhancement and subsequently increased biological reactions in the tissue. The underlying rationale for physical dose enhancement is that high-Z metal has a higher stopping power of radiation than the soft tissue. While the Compton effect, photoelectric effect, and pair production occur when radiation is irradiated to the matter, high-Z metal NPs can induce higher energy deposition to the cancer tissue. In non-limiting examples, metal nanoparticles for use in the present disclosure include, gold, platinum, gadolinium, silver, titanium, zinc, cerium, iron, thallium, and various metal oxides (non-limiting examples include NiO, ZnO, MnO₂, Fe₂O₃, TiO₂, and Co₃O₄). In exemplary embodiments, the metal nanoparticle is a gold or iron nanoparticle.

The nanoparticle can be any form of strontium aluminum oxide Sr_{w}AlₓO_{y} doped with a rare earth element (RaE) such as Eu²⁺, Dy³⁺, Nd³⁺, or combinations thereof, wherein the ratio of “y/x” is from 1 to 10 and/or the ratio “w/x” is from 1 to 10 (e.g., Sr₄Al₁₄O₂₅, SrAl₂O₄, SrAl₂O₇, and Sr₃Al₂O₆ doped with Eu²⁺, Dy³⁺, Nd³⁺, or combinations thereof). Examples of suitable nanoparticle material include, but are not limited to, any form of strontium aluminum oxide, such as SrₐAl_{b}O_{c}, where a, b, and c are integers that can vary; any form of strontium aluminum oxide doped with a rare earth element (RaE), SrₐAl_{b}O_{c}:RaE, wherein a, b, and c are integers that can vary and RaE is Ce, Pr, Nd, Sm, Eu, Tb, Dy, Ho, Er, Tm, or Yb in one or more oxidation states, such as Europium(II), Dysprosium(III), or Neodymium(III) doped Sr₄Al₁₄O₂₅, SrAl₂O₄, SrAl₂O₇, and Sr₃Al₂O₆; any form of strontium aluminum oxide co-doped with two or more different rare earth elements (RaEs), Sn aAl bO c:(RaE) 2, wherein a, b, and c are integers that can vary and RaE is Ce, Pr, Nd, Sm, Eu, Tb, Dy, Ho, Er, Tm, or Yb in one or more oxidation states, such as strontium aluminum oxide co-doped with Europium(II) and Dysprosium(III) as in Sr₄Al₁₄O₂₅:Eu²⁺:Dy³⁺, SrAl₂O₄:Eu²⁺:Dy³⁺, SrAl₂O₇:Eu²⁺:Dy³⁺, and Sr₃Al₂O₆:Eu²⁺:Dy³⁺; and strontium aluminum oxide co-doped with Europium(II) and Neodymium(III) as in Sr₄Al₁₄O₂₅:Eu²⁺:Nd³⁺, SrAl₂O₄:Eu²⁺:Nd³⁺, SrAl₂O₇:Eu²⁺Nd³⁺, and Sr₃Al₂O₆:Eu²⁺:Nd³⁺; any form of rare-earth ion-doped gadolinium oxide or oxysulfide phosphor, Gd₂O₃:RaE³⁺ or Gd₂O₂S:RaE³⁺, wherein RaE is Ce, Pr, Nd, Sm, Eu, Tb, Dy, Ho, Er, Tm, or Yb; any rare-earth (RaE) ion co-doped alkaline earth aluminum oxide, ₓMO⁺_{y}Al₂O₂:RaE, where x and y are integers, and M is Ca, Sr, or Ba, and RaE is Ce, Pr, Nd, Sm, Eu, Tb, Dy, Ho, Er, Tm, or Yb; any rare-earth- or transition-metal-doped metal halide, including, but not limited to, LaF₃:Ce³⁺, LuF₃:Ce³⁺, CaF₂:Mn²⁺, CaF₂:Eu²⁺, BaFBr:Eu²⁺, BaFBr:Mn²⁺, CaPO⁴:Mn²⁺, LuI³:Ce, SrI²:Eu, CaI²:Eu, GdI³:Ce; or any other suitable material, such as CdS, CdSe, CdTe, CaWO₄, ZnS:Cu, TmO, ZnSe:Te, ZnS, ZnO, TiO₂, GaN, GaAs, GaP, InAs, InP, Y₂O₃, WO₃, and ZrO₂. Specific examples of integers for index a can be 1, 2, 3, 4, 5, 6, 7, and 8. Specific examples of integers for index b can be 1, 2, 3, 4, 5, 6, 7, 8, 9, 10, 11, 12, 13, 14, 15, 16, 17, 18, 19, and 20. Specific examples of integers for index c can be 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30. These materials can be made by chemical synthesis, solid state reaction, other methods, or any combination thereof.

Representative nanoparticles that can be used in the disclosed photodynamic system include, but are not limited to, any form of strontium aluminum oxide doped with Europium(II), such as Sr₄Al₁₄O₂₅:Eu²⁺, SrAl₂O₄:Eu²⁺, SrAl₂O₇:Eu 2+, or Sr₃Al₂O₆:Eu²⁺. In some examples, the nanoparticle can be any strontium aluminum oxide co-doped with Europium(II) and Dysprosium(III).

In some further examples, the nanoparticle can be a semiconductor nanomaterial such as ZnS, ZnO, or TiO₂. Other examples of suitable scintillation nanoparticles include, but are not limited to, CaF₂, BaFBr, and CaPO₄, doped nanoparticles.

The nanoparticle can be a long-afterglow nanoparticle. These nanoparticles are luminescent materials with long decay lifetimes, ranging from a few minutes to tens of hours. Nanoparticles that exhibit both scintillation and afterglow luminescence can also be used with the presently disclosed photodynamic therapy system. When such “afterglow” nanoparticles are used in the photodynamic therapy system, the radiation dose can be greatly reduced. For example, if scintillation nanoparticles without afterglow are used, 30 seconds of radiation dosing may be used to generate enough photons for photodynamic therapy activation; whereas, if scintillation nanoparticles with afterglow are used, only 10 seconds of radiation dosing may be needed to generate enough photons for photodynamic therapy because extra photons are contributed from the afterglow. Therefore, the benefits and applications of nanoparticles having afterglow are tremendous.

In some examples, the nanoparticle can be biocompatible, such that the nanoparticle composition according to the disclosure is suitable for use in a variety of biological applications. “Biocompatible” or “biologically compatible”, as used herein, generally refer to compounds or particles that are, along with any metabolites or degradation products thereof, generally non-toxic to cells and tissues, and which do not cause any significant adverse effects to cells and tissues when cells and tissues are incubated (e.g., cultured) in their presence. Some biocompatible nanoparticles are nanoparticles that degrade hydrolytically into nontoxic byproducts. In some embodiments, the biocompatible nanoparticles can be water-soluble and stable in biological environments. Examples of suitable biocompatible nanoparticles include, but are not limited to, strontium aluminum oxide and calcium phosphate nanoparticles. Calcium phosphate nanoparticles are non-toxic and are being developed as a vaccine adjuvant and for targeted gene delivery.

Other nanoparticles with a certain level of toxicity, such as CdTe and CdSe nanoparticles can also be used. These nanoparticles can be surface coated with biocompatible material such as silica, alumina, titanium oxide or polymers in order to reduce their toxicity.

As described above, the nanoparticle can be an oxide, for example, aluminum oxide, zinc oxide, titanium oxide, zirconium oxide, strontium oxide, silicon oxide, cerium oxide, tin oxide, magnesium oxide, cadmium oxide, copper aluminum oxide, silver oxide, gallium oxide, tantalum oxide, thorium oxide, gold, silver, gadolinium oxide, ytterbium, stannic oxide, calcium tungstate, oxysulfide, cobalt ferrite, and combinations thereof.

In some embodiments, the emission wavelength and quantum yield of the nanoparticle can be modified by the geometric dimensions (size) of the nanoparticle. Therefore, in the present disclosure, the particle emission wavelength can be controlled to match the absorption band of the photosensitizers by controlling the geometric dimensions of the nanoparticle. The nanoparticle can have geometric dimensions from about 5 nm to about 5000 nm. For example, the nanoparticle can have a geometric dimension of less than about 10 nm, less than about 20 nm, less than about 30 nm, less than about 40 nm, less than about 50 nm, less than about 100 nm, less than about 200 nm, less than about 250 nm, less than about 300 nm, less than about 350 nm, less than about 400 nm, less than about 450 nm, less than about 500 nm, less than about 550 nm, less than about 600 nm, less than about 650 nm, less than about 700 nm, less than about 800 nm, less than about 900 nm, less than about 1000 nm, less than about 1500 nm, or less than about 2000 nm, greater than about 10 nm, greater than about 20 nm, greater than about 30 nm, greater than about 40 nm, greater than about 50 nm, greater than about 60 nm, greater than about 70 nm, greater than about 80 nm, greater than about 90 nm, greater than about 100 nm, greater than about 150 nm, greater than about 200 nm, greater than about 250 nm, greater than about 300 nm, greater than about 350 nm, greater than about 400 nm, greater than about 450 nm, greater than about 500 nm, greater than about 550 nm, greater than about 600 nm, greater than about 650 nm, greater than about 700 nm, greater than about 750 nm, greater than about 800 nm, greater than about 850 nm, greater than about 900 nm, greater than about 950 nm, greater than about 1000 nm, from about 1 nm to about 2000 nm, about 1 nm to about 1500 nm, about 1 nm to about 1000 nm, about 1 nm to about 750 nm, about 1 nm to about 500 nm, about 1 nm to about 300 nm, about 1 nm to about 100 nm, from about 5 nm to about 2000 nm, about 5 nm to about 1500 nm, about 5 nm to about 1000 nm, about 5 nm to about 750 nm, about 5 nm to about 500 nm, about 5 nm to about 300 nm, about 5 nm to about 100 nm, about 50 nm to about 2000 nm, about 50 nm to about 1000 nm, about 50 nm to about 750 nm, about 50 nm to about 650 nm, about 50 nm to about 500 nm, about 100 nm to about 1000 nm, about 100 nm to about 900 nm, about 100 nm to about 800 nm, about 100 nm to about 700 nm, about 100 nm to about 600 nm, about 100 nm to about 500 nm, about 100 nm to about 400 nm, about 200 nm to about 1000 nm, about 200 nm to about 850 nm, about 200 nm to about 750 nm, about 200 nm to about 700 nm, about 200 nm to about 650 nm, about 200 nm to about 600 nm, about 200 nm to about 500 nm, about 200 nm to about 400 nm, about 200 nm to about 350 nm, about 200 nm to about 300 nm, about 250 nm to about 800 nm, about 250 nm to about 750 nm, about 250 nm to about 700 nm, about 250 nm to about 650 nm, about 250 nm to about 600 nm, about 250 nm to about 550 nm, about 250 nm to about 500 nm, about 250 nm to about 450 nm, about 250 nm to about 400 nm, about 300 nm to about 1000 nm, about 300 nm to about 900 nm, about 300 nm to about 800 nm, about 300 nm to about 750 nm, about 300 nm to about 700 nm, about 300 nm to about 650 nm, about 300 nm to about 600 nm, about 300 nm to about 550 nm, about 300 nm to about 500 nm, about 300 nm to about 450 nm, about 300 nm to about 400 nm, or about 300 nm to about 350 nm. The nanoparticles can be spherical or asymmetric.

In some embodiments, the emission energy or wavelength can also be adjusted by the use of different dopants in the nanoparticle. The nanoparticle can absorb energy then emits at a preferred wavelength as a result of a dopant ion in the nanoparticle. The nanoparticle can be doped with at least one rare earth element or lanthanide such as La, Ce, Pr, Nd, Pm, Sm, Eu, Gd, Tb, Dy, Ho, Er, Tm, Yb, or Lu, any one if which can be at various oxidation states. The amount of dopant ion in the nanoparticle can be in an amount greater than about 0.1 wt %, greater than about 0.3 wt %, greater than about 0.5 wt %, greater than about 0.7 wt %, greater than about 0.9 wt %, greater than about 1 wt %, greater than about 1.5 wt %, greater than about 2 wt %, less than about 0.2 wt %, less than about 0.5 wt %, less than about 0.7 wt %, less than about 1 wt %, less than about 1.5 wt %, less than about 2 wt %, less than about 2.5 wt %, less than about 5 wt %, or less than about 10 wt %.

The emission energy can be further enhanced by dielectric confinement. If the dielectric constant (c) of the nanoparticles is greater than that of the surrounding matrix, the electric force lines of the particles will penetrate into the matrix, and the Coulomb interaction will be enhanced. As a consequence, the binding energy and the oscillator strength of the exciton are greatly increased. This is called dielectric confinement. This effect can be used to further enhance the emission energy and stability of the nanoparticles. ZnO (c=1.7) and SiO₂ (c=3.9) are suitable materials as their dielectric constants are lower than the CdS (c=9.12), ZnS (c=8.2), CaF₂ (c=6.76), BaFBr (c=14.17), and CaPO₄ (c=14.5) nanoparticles. Thus, when these nanoparticles are coated with ZnO or SiO₂ to form core/shell structures, they have very high luminescence quantum efficiencies as a result of quantum size confinement and dielectric confinement. In addition, coating with ZnO or SiO₂ can increase the stability and reduce the toxicity of the nanoparticles. However, the coating layer (shell) should be thinner than the energy transfer critical distance so that it does not block the energy transfer from the nanoparticles to the photosensitizers.

For example, the shell thickness can have a geometric dimension of less than about 5 nm, less than about 10 nm, less than about 20 nm, less than about 30 nm, less than about 40 nm, less than about 50 nm, less than about 100 nm, less than about 200 nm, less than about 250 nm, less than about 300 nm, less than about 350 nm, less than about 400 nm, less than about 450 nm, less than about 500 nm, less than about 550 nm, less than about 600 nm, less than about 650 nm, less than about 700 nm, less than about 800 nm, less than about 900 nm, less than about 1000 nm, less than about 1500 nm, or less than about 2000 nm, greater than about 10 nm, greater than about 20 nm, greater than about 30 nm, greater than about 40 nm, greater than about 50 nm, greater than about 60 nm, greater than about 70 nm, greater than about 80 nm, greater than about 90 nm, greater than about 100 nm, greater than about 150 nm, greater than about 200 nm, greater than about 250 nm, greater than about 300 nm, greater than about 350 nm, greater than about 400 nm, greater than about 450 nm, greater than about 500 nm, greater than about 550 nm, greater than about 600 nm, greater than about 650 nm, greater than about 700 nm, greater than about 750 nm, greater than about 800 nm, greater than about 850 nm, greater than about 900 nm, greater than about 950 nm, greater than about 1000 nm, from about 1 nm to about 2000 nm, about 1 nm to about 1500 nm, about 1 nm to about 1000 nm, about 1 nm to about 750 nm, about 1 nm to about 500 nm, about 1 nm to about 300 nm, about 1 nm to about 100 nm, about 50 nm to about 2000 nm, about 50 nm to about 1000 nm, about 50 nm to about 750 nm, about 50 nm to about 650 nm, about 50 nm to about 500 nm, about 100 nm to about 1000 nm, about 100 nm to about 900 nm, about 100 nm to about 800 nm, about 100 nm to about 700 nm, about 100 nm to about 600 nm, about 100 nm to about 500 nm, about 100 nm to about 400 nm, about 200 nm to about 1000 nm, about 200 nm to about 850 nm, about 200 nm to about 750 nm, about 200 nm to about 700 nm, about 200 nm to about 650 nm, about 200 nm to about 600 nm, about 200 nm to about 500 nm, about 200 nm to about 400 nm, about 200 nm to about 350 nm, about 200 nm to about 300 nm, about 250 nm to about 800 nm, about 250 nm to about 750 nm, about 250 nm to about 700 nm, about 250 nm to about 650 nm, about 250 nm to about 600 nm, about 250 nm to about 550 nm, about 250 nm to about 500 nm, about 250 nm to about 450 nm, about 250 nm to about 400 nm, about 300 nm to about 1000 nm, about 300 nm to about 900 nm, about 300 nm to about 800 nm, about 300 nm to about 750 nm, about 300 nm to about 700 nm, about 300 nm to about 650 nm, about 300 nm to about 600 nm, about 300 nm to about 550 nm, about 300 nm to about 500 nm, about 300 nm to about 450 nm, about 300 nm to about 400 nm, or about 300 nm to about 350 nm.

A nanoparticle according to the disclosure can have coating layer comprising a biocompatible polymer conjugated to the surface of the nanoparticle. For example, the coating layer (shell) can be polyethylene glycol (PEG) group(s) disposed on (e.g., covalently bonded to) a surface of the nanoparticle. In an example, at least a portion of the exterior surface (e.g., at least 20%, 30%, 40% or 50% of the exterior surface) of a nanoparticle is functionalized with polyethylene glycol groups. In some embodiments, when present, the coating layer facilitates conjugation of the photosensitizer to the nanoparticle, for example, through a covalent bond. In various examples, the number of PEG group(s) disposed on the surface of a nanoparticle is 3 to 600, including all integer number of PEG group(s) and ranges there between. In some aspects, a nanoparticle of the disclosure is coated with unmodified poly(ethylene)glycol (PEG) molecules of different molecular weights and surface charges. Other non-limiting examples of biocompatible surface coating include poly dopamine and chitosan

In the present description, the term “polyethylene glycol” is understood to be any hydrophilic polymer soluble in water containing ether groups linked by 2 or 3 carbon atom, optionally branched alkylene groups. Therefore this definition includes branched or non-branched polyethylene glycols, polypropylene glycols, and also block or random copolymers including the two types of units. The term also includes derivatives of the terminal hydroxyl groups, which can be modified (1 or both ends) so as to introduce alkoxy, acrylate, methacrylate, alkyl, amino, phosphate, isothiocyanate, sulfhydryl, mercapto and sulfate groups. The polyethylene glycol or polypropylene glycol can have substituents in the alkylene groups. If they are present, these substituents are preferably alkyl groups.

Polyethylene glycols are water-soluble polymers that have been approved for the oral, parenteral and topical administration of drugs (FDA). Polyethylene glycols are produced by means of polymerization of ethylene oxide (EO) or propylene oxide (PO) in the presence of water, monoethylene glycol or diethylene glycol as reaction initiators in an alkaline medium (1,2-Epoxide Polymers: Ethylene Oxide Polymers and Copolymers” in Encyclopedia of Polymer Science and Engineering; Mark, H. F. (Ed.), John Wiley and Sons Inc., 1986, pp. 225-273). When the desired molecular weight (generally controlled by means of in-process measurements of viscosity) is reached, the polymerization reaction ends by neutralizing the catalyst with an acid (lactic acid, acetic acid or the like). The result is a linear polymer having a very simple structure: HO—(CH₂—CH₂—O)n-H; where (n) is the number of EO monomers or units. The units alternatively contain propylene groups. Methods of generating PEGylated nanoparticles are known in the art.

One aspect of the present disclosure encompasses a photosensitizer conjugated to a gold nanoparticle. In some embodiments, the gold nanoparticle comprises a polyethylene glycol coated (PEGylated). In an aspect, the photosensitizer is covalently attached to the PEG coating.

In some embodiments, the disclosure provides a nanoparticle functionalized with various groups. The groups may be covalently bound to a surface of the nanoparticle and/or part of a PEG group covalently bound to a surface of the nanoparticle. For example, a nanoparticle is functionalized with groups chosen from peptides (natural or synthetic), cyclic peptides (e.g., cyclic-RGD and derivatives thereof, alpha-MSH and derivatives thereof, and the like), nucleic acids (e.g., single stranded or double stranded DNA, various forms of RNA (e.g., siRNA, and the like), lipids, carboyhydrates (e.g., oligosaccharides, polysaccharides, sugars, and the like), groups comprising a radio label (e.g., ¹²⁴I, ¹³¹I, ²²⁵Ac or ¹⁷⁷Lu, ⁸⁹Zr, ⁶⁴Cu, and the like), antibodies, antibody fragments, groups comprising a reactive group (e.g., a reactive group that can be further conjugated, for example, via click chemistry, to a molecule such as, for example, a pharmaceutical product (e.g., a drug molecule, which may be a toxic drug molecule, a small molecule inhibitor (e.g., gefitinib, and the like)), and combinations thereof.

At least a portion of an exterior surface of a nanoparticle may be functionalized with at least one targeting moiety. A nanoparticle can have various amounts of targeting moieties. For example, a nanoparticle has 1-50 targeting moieties disposed on (e.g., covalently bonded to) an exterior surface of the nanoparticle. In various examples, a nanoparticle has 1-3 targeting moieties, 1-10 targeting moieties, 1-20 targeting moieties, or 1-40 targeting moieties disposed on (e.g., covalently bonded to) an exterior surface of the nanoparticle.

The specificity of the disclosed nanoparticle composition can be increased by conjugation of the system with a targeting moiety, which, in some embodiment, specifically binds to a component on the surface of, for example, a target cell or tissue. Target recognition moiety includes cell recognition moieties which specifically bind to receptors on the surface of a target cell. Steinberg, E. D., et al., Tetrahedron, 54, 4151-4202 (1998) discloses the design of new generations of photosensitizers for the treatment of tumors, the disclosure of which is incorporated herein by reference in its entirety for teachings of various cell recognition moieties. In the disclosed compositions, the cell recognition moiety can typically be present on the nanoparticle composition, e.g., the protein cage.

A wide variety of natural and synthetic molecules recognized by target cells can be used as the cell recognition moiety. Suitable cell recognition moieties include, but are not limited to, a receptor, ligand, polynucleotide, peptide, polynucleotide binding agent, antigen, antibody, or combinations thereof. In one embodiment, the cell recognition moiety is a peptide which has a length of from about 6 amino acids to about 25 amino acids.

The targeting moiety, for example the peptide amino acid sequence, can be similar, homologous, or a variant of targeting moieties in the art. In general, variants of the cell targeting moiety for example nucleic acids and peptides herein disclosed, can have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% similarity, or homology, to the stated sequence or the native sequence. Those of skill in the art readily understand how to determine the similarity of two polypeptides or nucleic acids. For example, the similarity can be calculated after aligning the two sequences so that the similarity is at its highest level. As an example, peptides can have one or more conservative amino acid substitutions. These conservative substitutions are such that a naturally occurring amino acid is replaced by one having similar properties. Such conservative substitutions do not alter the function of the peptide.

The following references discloses antibodies, receptors, or receptor ligands that can be used to target specific proteins to tumor tissue: (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)), disclosure of which are incorporated herein by reference. The following references discloses vehicles such as “stealth” and other antibody conjugated particles (including lipid mediated drug targeting to colonic carcinoma), receptor mediated targeting through cell specific ligands, lymphocyte directed tumor targeting, and highly specific therapeutic retroviral targeting of murine glioma cells in vivo: (Hughes et al., Cancer Research, 49:6214-6220, (1989); and Litzinger and Huang, Biochimica et Biophysica Acta, 1104:179-187, (1992)), disclosure of which are incorporated herein by reference.

In some embodiments, a nanoparticle compositions according to the disclosure include a ligand conjugated to the coating of the nanoparticle. Ligands useful according to the present disclosure include, but are not limited to, diagnostic and/or therapeutic agents (e.g., drugs). Examples of therapeutic agents include, but are not limited to, chemotherapeutic agents, antibiotics, antifungal agents, antiparasitic agents, antiviral agents, and combinations thereof. An affinity ligand may be also be conjugated to the nanoparticle to allow targeted delivery of the nanoparticles. For example, the nanoparticle may be conjugated to a ligand which is capable of binding to a cellular component (e.g., on the cell membrane or in the intracellular compartment) associated with a specific cell type. The targeted molecule may be a tumor marker or a molecule in a signaling pathway. The ligand can have specific binding affinity to certain cell types, such as, for example, tumor cells. In certain examples, the ligand may be used for guiding the nanoparticles to specific areas, such as, for example, liver, spleen, brain or the like. Imaging can be used to determine the location of the nanoparticles in an individual. Examples of diagnostic agents include fluorescent dyes. Examples of fluorescent dyes and conjugation methods for fluorescent dyes are known in the art.

For example, a drug-linker conjugate, where the linker group can be specifically cleaved by enzyme or acid condition in tumor for drug release, may be covalently attached to the functional ligands on the particles for drug delivery. For example, drug-linker-thiol conjugates are attached to maleimido-PEG-particles through thiol-maleimido conjugation reaction post the synthesis of maleimido-PEG-particles. Additionally, both drug-linker conjugate and cancer targeting peptides may be attached to the particle surface for drug delivery specifically to tumor.

A ligand may be a biomolecule. Non-limiting examples of biomolecules include biotin, targeting ligands (e.g., targeting peptides, which may be natural or synthetic peptides, such as, for example, cyclic-RGD and derivatives thereof, alpha-MSH and derivatives thereof, and the like), targeting antibody or antibody fragments, targeting glycans (e.g., sugar molecules targeting cell surface receptors), nucleic acids (e.g., single stranded or double stranded DNA, various forms of RNA (e.g., siRNA, and the like), lipids, and carboyhydrates (e.g., oligosaccharides, polysaccharides, sugars, and the like). A ligand may be a chelator molecule for metal radioisotopes, such as, for example, deferoxamine (DFO), which is an efficient chelator for radio-labeling with, for example, Zr⁸⁹, NODA, DOTA, drug molecules, and the like. A chelator molecule can form a chelating moiety that binds a radio metal (e.g., radio label) to a nanoparticle. Nanoparticles with radio metals may be used to perform PET or radiotherapy. Nanoparticles with a drug molecule/molecules may be used in therapeutic methods.

As described above, a nanoparticle according to the disclosure contain a photosensitizer useful for causing photodynamic damage cells. Damage, as used herein, includes destruction of cellular organelles and subsequently suppression of cell growth, suppression of cell growth rate, and/or cell death. In some examples, the emission spectra of the nanoparticles can be matched to the absorption spectra of the photosensitizers. Upon absorption of electromagnetic radiation, the photosensitizer molecules are excited to a short-lived singlet state. Following excitation, fast radiationless relaxation to the lower-lying triplet states occurs via intersystem crossing and ultimately yields the first excited triplet state Ti in a spin-allowed process. The longer the decay lifetime of the triplet state, the more time the photosensitizer has to act on a tissue, such as a tumor tissue and to initiate biochemical and biophysical mechanisms, which cause tumor necrosis. Therefore a long triplet lifetime (>500 ns) can be considered a precondition for efficient photosensitization.

The photosensitizer is bound to the nanoparticle through a coordinate bond. In some embodiments, the photosensitizer has a zero-length conjugation to the surface of the nanoparticle. As used herein, the term “zero-length conjugation” refers to the conjugation of two molecules by forming a bond containing no additional atoms. Thus, one atom of a molecule is covalently attached to an atom of a second molecule with no intervening linker or spacer. In some embodiments, the photosensitizer comprises a carboxylate, thiol, hydroxy, amino or phosphate group; the nanoparticle comprises a metal; and the carboxylate, thiol, hydroxyl, amino or phosphate group is bound to the metal. In some embodiments, the photosensitizer comprises a carboxylate, thiol, hydroxy, amino or phosphate group; the nanoparticle comprises a metal; and the carboxylate, thiol, hydroxyl, amino or phosphate group is bound to the metal. In some embodiments, the photosensitizer and the nanoparticle are linked and the linkage comprises a cyclodextrin, polyethylene glycol, poly(maleic acid), or a C₂-C₁₅ linear or branched alkyl chain.

The term “photosensitizer” (PS) refers to a chemical compound or moiety that can be excited by light of a particular wavelength, typically visible or near-infrared (NIR) light, and produce a reactive oxygen species (ROS). For example, in its excited state, the photosensitizer can undergo intersystem crossing and transfer energy to oxygen (O₂) (e.g., in tissues being treated by PDT) to produce ROSs, such as singlet oxygen (¹O₂). Any known type of a photosensitizer can be used in accordance with the presently disclosed subject matter. In some embodiments, the photosensitizer is a porphyrin, a chlorophyll, a dye, or a derivative or analog thereof. In some embodiments, phophyrins, chlorins, bacteriochlorins, or porphycenes can be used. In some embodiments, the photosensitizer can have one or more functional groups, such as carboxylic acid, amine, or isothiocyanate, e.g., for using in attaching the photosensitizer to another molecule or moiety, such as an organic bridging ligand or a SBU, and/or for providing an additional site or sites to enhance coordination or to coordinate an additional metal or metals. In some embodiments, the photosensitizer is a porphyrin or a derivative or analog thereof. Exemplary porphyrins include, but are not limited to, hematoporphyrin, protoporphyrin and tetraphenylporphyrin (TPP). Exemplary porphyrin derivatives include, but are not limited to, pyropheophorbides, bacteriochlorophylls, chlorophyll a, benzoporphyrin derivatives, tetrahydroxyphenyl chlorins, purpurins, benzochlorins, naphthochlorins, verdins, rhodins, oxochlorins, azachlorins, bacteriochlorins, tolyporphyrins and benzobacteriochlorins. Porphyrin analogs include, but are not limited to, expanded porphyrin family members (such as texaphyrins, sapphyrins and hexaphyrins), porphyrin isomers (such as porphycenes, inverted porphyrins, phthalocyanines, and naphthalocyanines), and TPP substituted with one or more functional groups.

The photosensitizer can be a macrocyclic organic complex, which absorbs radiation in the range of from about 300 nm to about 900 nm, typically from about 400 nm to about 800 nm. These photosensitizers are capable of transferring their absorbed energy to molecular oxygen to generate singlet oxygen. Examples of suitable macrocyclic organic complexes include, but are not limited to, porphyrin and their derivatives, pyrrole, tetrapyrollic compound, expanded pyrrolic macrocycle and their derivatives, cyanine and their derivatives, flavin, organometallic species, nanoparticle, or combinations thereof. Representative examples of suitable macrocyclic compounds include, but are not limited to, green porphyrins, protoporphyrin, chlorins, tetrahydrochlorins (chlorins bacteriochlorins, isobacteriochlorins), hematoporphyrin, benzoporphyrin, texaohyrins, chlorophylls, dyes, aminolevulinic acid (ALA), silicon phthalocyanine Pc 4, m-tetrahydroxyphenylchlorine (mTHPC), mono-L-aspartyl chlorine (Npe6), pyropheophosphides, purpurins, texaphyrins, phenothiaziniums, phthalocyanines, napthalocyanines, porphycenes, pheophorbides, merocyanine, phthalocyanine, chloroaluminum phthalocyanine, sulfonated aluminum phthalocyanine, ring-substituted cationic phthalocyanine, disulfonated or tetrasulfonated derivative, sulfonated aluminum naphthalocyanine, naphthalocyanine, tetracyanoethylene adduct, crystal violet, azure β chloride, benzophenothiazinium, benzophenothiazinium chloride (EtNBS), phenothiazine derivative, rose Bengal, toluidine blue derviatives, toluidine blue O (TBO), methylene blue (MB), new methylene blue N (NMMB), new methylene blue BB, new methylene blue FR, 1,9-dimethylmethylene blue chloride (DMMB), methylene blue derivatives, methylene green, methylene violet Bernthsen, methylene violet 3RAX, Nile blue, Nile blue derivatives, malachite green, Azure blue A, Azure blue B, Azure blue C, safranine O, neutral red, 5-ethylamino-9-diethylaminobenzo[a]phenothiazinium chloride, 5-ethylamino-9-diethylaminobenzo[a]phenoselenazinium chloride, thiopyronine, thionine, or combination thereof. Photosensitizers currently approved by the FDA for photodynamic therapy, such as Photofyrin (actually a mixture of porphyrins, including photoporphyrin, haematoporphyrin, hydroxyethyldeuteropophyrin); and verteporfin, a benzoporphyrin, can also be used in the compositions. In an exemplary embodiment, the photosensitizer is chlorin e6.

The photosensitizers can contain metal cations. The metal ion present in the photosensitizer can be a diamagnetic metal. The metal ion present in the photosensitizer can be a diamagnetic metal. Suitable diamagnetic metals include, but are not limited to aluminum, copper, zinc, tin, silicon, germanium, lithium, magnesium, platinum, palladium, iridium, rudinium, ruthenium, rhenium, osmium, technetium, and combinations thereof. Suitable examples of metal-containing photosensitizers include, but are not limited to, zinc phthalocyanine, sulfonated aluminum phthalocyanine, and magnesium phthalocyanine, and zinc tetraphenyl porphyrin.

Some nanoparticles can be photoactivated to produce singlet oxygen. These photoactivated nanoparticles can be used in the compositions. Nanoparticle photosensitizers have some advantages in that, they can be made hydrophilic, they possess relatively large surface area, owing to their sub-cellular and nanometer size, nanoparticles can penetrate deep into tissue through fine capillaries and pass through the fenestrae into the epithelial lining so that they can be taken up efficiently by cells, they have high extinction or absorption coefficients, and they are photostable for in vivo applications. Examples of suitable nanoparticles that can be used as a photosensitizer include, but are not limited to, CdTe, CdS, ZnO, TiO₂, and Si nanoparticles.

The selection of nanoparticle and photosensitizer can be in a manner to promote energy transfer from the nanoparticles to the photosensitizers thereby ensuring efficient photoactivation. In some embodiments, the energy transfer between the nanoparticle and photosensitizer can be via fluorescence resonance energy transfer (FRET). As used here, FRET refers to the transfer from the initially excited donor (the scintillation nanoparticle) to an acceptor (the photosensitizer). For efficient energy transfer, the emission band of the donor must overlap effectively with the absorption band of the acceptor, and/or the donor and the acceptor must be close enough spatially to permit transfer. FRET energy transfer rate is highly dependent on the distance between the donor and receptor. The distance at which FRET is 50% efficient is called the Förster distance, typically about 2-10 nm. Generally, in order to have an efficient energy transfer, the distance between the donor and the acceptor may be less than about 10 nm.

A nanoparticle or plurality of nanoparticles can exhibit desirable properties. For example, a nanoparticle or plurality of nanoparticles exhibit an increase of the singlet oxygen quantum yield, relative to the free photosensitizer(s) used in the nanoparticles in solution (e.g., aqueous solution), of 10% to 1000%, including all integer % values and ranges there between. In various examples, a nanoparticle or plurality of nanoparticles exhibit an increase of the singlet oxygen quantum yield, relative to the free photosensitizers(s) used in the nanoparticles in solution (e.g., aqueous solution), of 10% or more, 20% or more 30% or more, 40% or more, 50% or more, 75% or more, 100% or more, 250% or more, 500% or more, or 1000% or more.

b) Components of the Composition

The present disclosure also provides pharmaceutical compositions. The pharmaceutical composition comprises a nanoparticle composition of the present disclosure, as an active ingredient, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In each of the embodiments described herein, a composition of the invention may optionally comprise one or more additional drug or therapeutically active agent in addition to the nanoparticle composition of the present disclosure. Thus, in addition to the therapies described herein, one may also provide to the subject other therapies known to be efficacious for treatment of a tumor or cancer. In some embodiments, the secondary agent is selected from a cancer related-chemotherapeutic agent, radiation, corticosteroid, a non-steroidal anti-inflammatory drug (NSAID), an intravenous immunoglobulin, a kinase inhibitor, a fusion or recombinant protein, a monoclonal antibody, or a combination thereof. In some embodiments, agents suitable for combination therapy include but are not limited to inhaled bronchodilators and inhaled steroids.

(i) Diluent

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

(ii) Binder

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

(iii) Filler

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

(iv) Buffering Agent

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

(v) pH Modifier

In various embodiments, the excipient may be a pH modifier. By way of non-limiting example, the pH modifying agent may be sodium carbonate, sodium bicarbonate, sodium citrate, citric acid, or phosphoric acid.

(vi) Disintegrant

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

(vii) Dispersant

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

(viii) Excipient

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

(ix) Lubricant

In a further embodiment, the excipient may be a lubricant. Non-limiting examples of suitable lubricants include minerals such as talc or silica; and fats such as vegetable stearin, magnesium stearate, or stearic acid.

(x) Taste-Masking Agent

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

(xi) Flavoring Agent

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

(xii) Coloring Agent

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Additional formulations of pharmaceutical delivery systems may be in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners. A suitable pharmaceutically acceptable carrier to maintain optimum stability, shelf-life, efficacy, and function of the delivery system would be apparent to one of ordinary skill in the art.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition. In various examples, a method further comprises administering to the patient an additional cancer treatment. In some examples, the additional cancer treatment is chosen from the group comprising surgery, radiotherapy, chemotherapy, toxin therapy, immunotherapy, cryotherapy, gene therapy, and combinations thereof. In an example, a PDT method further comprises administration of a chemotherapy agent. In various examples, a chemotherapy agent is a drug or drug formulation. Non-limiting examples of drug formulations include polymeric micelle formulations, liposomal formulations, dendrimer formulations, polymer-based nanoparticle formulations, silica-based nanoparticle formulations, nanoscale coordination polymer formulations, nanoscale metal-organic framework formulations, inorganic nanoparticle formulations, and the like.

Various chemotherapy agents (e.g., chemotherapy drugs) can be used. Any FDA approved chemotherapy agent (e.g., chemotherapy drugs) can be used. Combinations of chemotherapy agents may be used.

c) Administration

The composition can be formulated into various dosage forms and administered by a number of different means that will deliver a therapeutically effective amount of the active ingredient. Such compositions can be administered orally, or parenterally in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

For parenteral administration (including subcutaneous, intraocular, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

Generally, a safe and effective amount of a nanoparticle composition is administered, for example, that amount that would cause the desired therapeutic effect in a subject while minimizing undesired side effects. In various embodiments, an effective amount of a nanoparticle composition described herein can substantially reduce viral infectivity in a subject suffering from a viral infection. In some embodiments, an effective amount is an amount capable of treating a respiratory viral infection. In some embodiments, an effective amount is an amount capable of treating one or more symptoms associated with a respiratory viral infection.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

The concentration of the nanoparticle of the present disclosure in the fluid pharmaceutical formulations can vary widely, i.e., from less than about 0.05% usually or at least about 2-10% to as much as 30 to 50% by weight and will be selected primarily by fluid volumes, viscosities, etc., in accordance with the particular mode of administration selected. For example, the concentration may be increased to lower the fluid load associated with treatment. The amount of nanoparticle pharmaceutical composition administered will depend upon the particular therapeutic entity entrapped inside the nanoparticle, the type of nanoparticle being used, and the judgment of the clinician. Generally the amount of nanoparticle pharmaceutical composition administered will be sufficient to deliver a therapeutically effective dose of the particular therapeutic entity.

The quantity of nanoparticle pharmaceutical composition necessary to deliver a therapeutically effective dose can be determined by routine in vitro and in vivo methods, common in the art of drug testing. See, for example, D. B. Budman, A. H. Calvert, E. K. Rowinsky (editors). Handbook of Anticancer Drug Development, LVVW, 2003. Therapeutically effective dosages for various therapeutic entities are well known to those of skill in the art; and according to the present disclosure a therapeutic entity delivered via the pharmaceutical liposome composition of the present invention provides at least the same, or 2-fold, 4-fold, or 10-fold higher activity than the activity obtained by administering the same amount of the therapeutic entity in its routine non-liposome formulation. Typically the dosages for the nanoparticle pharmaceutical composition of the present disclosure range between about 0.005 and about 500 mg of the therapeutic entity per kilogram of body weight, most often, between about 0.1 and about 100 mg therapeutic entity/kg of body weight.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Administration of a nanoparticle composition can occur as a single event or over a time course of treatment. For example, one or more of a nanoparticle composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a respiratory virus.

The present disclosure encompasses pharmaceutical compositions comprising compounds as disclosed above, so as to facilitate administration and promote stability of the active agent. For example, a compound of this disclosure may be admixed with at least one pharmaceutically acceptable carrier or excipient resulting in a pharmaceutical composition which is capably and effectively administered (given) to a living subject, such as to a suitable subject (i.e. “a subject in need of treatment” or “a subject in need thereof”). For the purposes of the aspects and embodiments of the invention, the subject may be a human or any other animal.

### II. Methods

The present disclosure relates, in general, to a method for administering a nanoparticle mediated-photodynamic therapy. In one embodiment, this method generally includes the steps of providing at least one nanoparticle composition as described herein; administering the nanoparticle composition to a subject or cell and administering an excitation source such that the nanoparticle emits electromagnetic radiation having a first wavelength when irradiated with the excitation source having electromagnetic radiation having a second wavelength (e.g. visible light, near-infrared light, and X-ray). In this embodiment, a photosensitizer attached to the nanoparticle absorbs the electromagnetic radiation having a first wavelength thereby providing the photodynamic therapy.

The methods and compositions comprising them, can be administered to an individual to kill endogenous tissue or cells. The tissue can be undesirable tissue that has arisen due to transformation, such as a tumor, cancer, or endometriosis; adipose tissue; plaques present in vascular tissue and over-proliferation such as those formed in restenosis; birthmarks and other vascular lesions of the skin; scars and adhesions; and irregularities in connective tissue or bone, such as bone spurs. As used herein, the term “cancer” includes a wide variety of malignant solid neoplasms. These can be caused by viral infection, naturally occurring transformation, or exposure to environmental agents. Parasitic infections and infections with organisms, especially fungal, that lead to disease may also be targeted. The compositions can also be used to permeabilize the endothelium and/or vasculature system in tumors to improve the enhanced permeable and retention (EPR) effect in tumor cells.

In some examples, the methods can be useful for causing photodynamic damage to cancer cells. Photodynamic damages to cancer cells include, but are not limited to, preventing or reducing the development of a cancer, reducing the symptoms of cancer, suppressing or inhibiting the growth of an established cancer, preventing metastasis and/or invasion of an existing cancer, promoting or inducing regression of the cancer, inhibiting or suppressing the proliferation of cancerous cells, reducing angiogenesis or increasing the amount of apoptotic cancer cells, thereby treating cancer.

Generally, the methods can include contacting a cell with an effective amount of the nanoparticle composition or a pharmaceutical composition as described herein. One of skill in the art recognizes that an amount can be considered therapeutically effective even if the condition is not totally eradicated but improved partially. The compositions can be injected directly into the target tissue, or can be administered systemically. More specifically, the compositions can be administered using any suitable method including intravenous (i.v.), intraperitoneal (i.p.), intramuscular (i.m.), intratumoral (i.t), intraarterial (i.a.), topically, and/or inhalation. Intravenous administration is particularly preferred for solid tumors, while i.p. administration is preferred for pancreatic, liver, and gastric tumors. Advantageously, even when administered systemically, the compositions preferentially accumulate in the cancerous tissue, and preferably actively integrate in the cancerous tissue, as opposed to surrounding healthy tissue.

The disclosed methods can also include the application of external ionizing radiation for the purpose of exciting the core of the nanoparticle. The rate and time the cancerous cells are irradiated may depend on the results required. For example, the cells can be irradiated at an effective fluence rate and time to cause permeabilization of the endothelial lining of the cancerous cells, i.e., increase in the Enhanced Permeabilization and Retention (EPR) effect without causing significant occlusion and/or collapse to tumor blood vessels. The cancerous cells can be irradiated at an effective fluence rate and time to cause therapeutic injury resulting in the reduction of at least one of the surface area, the depth, and the amount of the tissue affected by the cancerous condition. The irradiation regime may also be dependent on the compositions and design of the nanoparticle core and shell, the maximum safe dose of radiation that can be tolerated by the patient, or the targeted cell or material.

Irradiation can be any form of excitation radiation, including high-energy particles and radiation from all regions of the electromagnetic spectrum; ultrasound, electric fields and magnetic fields. In some embodiments, irradiation can be carried out using X-ray. X-ray is an energy source widely used in the clinic for both diagnosis and therapy purposes. X-ray can be given to cover either a small area (e.g. in dental radiography) or a large area (chest X-ray and CT). Both types may be employed herein. While narrow-beam X-ray can induce more focal and selective damage, X-ray covering a large area can permit the disclosed system to treat tumors of multiple loci or tumor metastasis. X-rays are advantageous because of both their ability to penetrate through the entire body and the amount of energy contained within the x-ray photon. The X-ray wavelengths can be less than about 10 nanometers, or from about 10 to about 0.01 nm. The power/fluence rate can be about 1 Sv/h or less. Typically, the fluence rate is from about 0.5 Sv/hr to about 1 Sv/hr. The cancerous/tumor cells can be irradiated for any period from about 5 minutes to about 60 minutes, or from about 15 minutes to about 30 minutes. X-ray devices that may be used in the methods herein include conventional commercial x-ray units commonly used for diagnostic or therapeutic purposes, computed-tomography (CT) scanners, full-body scanners or even X-ray lasers.

Other high-energy sources, such as gamma rays, and high-energy particles can also be used. A suitable range of gamma-ray radiation is an amount sufficient to pierce the human body and excite the nanoparticle material. Electromagnetic radiation in the wavelength range of 0.01 to 0.00001 nm is typically considered gamma radiation. High-energy particles include positrons, such as those used in positron emission tomography (PET) scans, and high-energy protons and electrons and are useful as external sources of energy.

The methods can also include the transfer of energy from the nanoparticle to the photosensitizer. One method of such energy transfer can be frequency resonance energy transfer (FRET), which is achieved when the emission spectrum of the core material overlaps the absorption spectrum of the photosensitizer, allowing plasmon excitation.

In some examples, the methods can include removing the nanoparticle or portion thereof, from the body. In such cases, the nanoparticle can be decorated or doped with magnetic material, typically on the surface, to allow magnetic removal of the particle from the blood by established cell-separation techniques.

In one example, the methods include administering a photodynamic therapeutic composition comprising a biocompatible nanoparticle that emits light having a first wavelength when irradiated with electromagnetic radiation (e.g. visible light, near-infrared light, and X-ray), a photosensitizer which absorbs light of said first wavelength, wherein the photosensitizer is attached to a PEG coating of the nanoparticle; and illuminating the treatment area by irradiation thereby causing the nanoparticles to emit light of the first wavelength.

In some embodiments, the combined treatment effects (i.e., the radiation and the photodynamic therapy will combine to kill cells. The combination of the photodynamic therapy and radiation results in supra-additive or synergistic effect in the killing of cancer or tumor cells. For example, the immune response to radiation-induced killing is different that the immunity generated by PDT-related cell killing and has significant implications for improved therapeutic results.

In an aspect, the present disclosure encompasses administering a therapeutically effective amount of a nanoparticle disclosed herein to a subject in need thereof. Suitable nanoparticles are described in detail in Section I. As used herein, the phrase “a subject in need thereof” refers to a subject in need of preventative or therapeutic treatment. A subject may be a rodent, a human, a livestock animal, a companion animal, or a zoological animal. In one embodiment, a subject may be a rodent, e.g., a mouse, a rat, a guinea pig, etc. In another embodiment, a subject may be a livestock animal. Non-limiting examples of suitable livestock animals may include pigs, cows, horses, goats, sheep, llamas and alpacas. In still another embodiment, a subject may be a companion animal. Non-limiting examples of companion animals may include pets such as dogs, cats, rabbits, and birds. In yet another embodiment, a subject may be a zoological animal. As used herein, a “zoological animal” refers to an animal that may be found in a zoo. Such animals may include non-human primates, large cats, wolves, and bears. In a preferred embodiment, a subject is a mouse. In another preferred embodiment, a subject is a human.

In another aspect, the present disclosure provides a method of killing a cancer or tumor cell, the method comprising contacting the cancer or tumor cell with an effective amount of a nanoparticle and excitation source as disclosed herein. In various embodiments, contact with the disclosed nanoparticles and excitation source results in energy transfer from the nanoparticles to the photosensitizers and the subsequent generation of singlet oxygen which are needed for effective cancer treatment. As an example, the details of how to realize and observe energy transfer from scintillation nanoparticles to photosensitizers and the generation of singlet oxygen are herein set forth. Efficient energy transfer from the scintillation nanoparticles to the photosensitizers is prerequisite for the generation of singlet oxygen for PDT. Two methods can be used to study and measure the energy transfer. With luminescence quenching, the luminescence efficiency or intensity of the scintillation nanoparticle is quenched when the photosensitizers are attached to the particles as energy transfers from the scintillation nanoparticles to the photosensitizers. This is a simple and direct method to study energy transfer between nanoparticles or between fluorophors.

Contacting a cancer cell with an effective amount of a disclosed nanoparticle involves admixing the delivery system and the cancer cell for a period of time sufficient to allow the concentration of the nanoparticle-photosensitizer in and/or around the cancer cell. This may occur in vitro or ex vivo or in vivo. The term “effective amount”, as used herein, means an amount that leads to measurable effect, e.g., cancer cell death. The effective amount may be determined by using the methods known in the art and/or described in further detail in the examples.

In another aspect, the present disclosure provides a method for treating a subject having a cancer or tumor. The method comprises administering to the subject a therapeutically effective amount of a nanoparticle disclosed herein to the subject. Suitable delivery systems are described in detail in Section I.

In some embodiments, the methods disclosed herein may further comprise obtaining a biological sample from a subject and assaying the biological sample to measure a signal from an imaging agent as disclosed herein. As used herein, the term “biological sample” may be, in non-limiting examples, a biological fluid, a tissue, a tissue homogenate, cells, a cellular lysate, a tissue or cell biopsy, skin cells, tumor or cancer cells, or any combination thereof. Any biological sample containing the disclosed delivery system is suitable. Numerous types of biological samples are known in the art. In some embodiments, the biological sample is a tissue sample such as a tissue biopsy. In one aspect, the biopsied tissue may be processed into individual cells or an explant, or processed into a homogenate, a cell extract, a membranous fraction, or a ceramide extract. In other embodiments, the sample may be a bodily fluid. Non-limiting examples of suitable bodily fluids include blood, plasma, serum, urine, and saliva. In a specific embodiment, the biological sample is blood, plasma, or serum. In a specific embodiment, the biological sample is plasma. The fluid may be used “as is”, the cellular components may be isolated from the fluid, or a fraction may be isolated from the fluid using standard techniques.

One of skill in the art will recognize that the amount and concentration of the composition administered to a subject will depend in part on the subject and the reason for the administration. Methods for determining optimal amounts are known in the art. Generally, a safe and effective amount of a delivery system composition is, for example, that amount that would cause the desired therapeutic effect in a subject while minimizing undesired side effects. In various embodiments, an effective amount of a delivery system composition described herein can substantially inhibit cancer progression, slow the progress of cancer, or limit the development of cancer.

The administration of a nanoparticle of the present disclosure can be carried out by aerosol inhalation, injection, ingestion, transfusion, implantation or transplantation. The delivery system compositions described herein, may be administered to a subject subcutaneously, intradermally, intratumorally, intranodally, intramedullary, intramuscularly, by intravenous or intralymphatic injection, or intraperitoneally. In one embodiment, the cell compositions of the present disclosure are preferably administered by intravenous injection.

Compositions of the disclosure are typically administered to a subject in need thereof in an amount sufficient to provide a benefit to the subject. This amount is defined as a “therapeutically effective amount.” A therapeutically effective amount may be determined by the efficacy or potency of the particular composition, the disorder being treated, the duration or frequency of administration, the method of administration, and the size and condition of the subject, including that subject's particular treatment response. A therapeutically effective amount may be determined using methods known in the art, and may be determined experimentally, derived from therapeutically effective amounts determined in model animals such as the mouse, or a combination thereof. Additionally, the route of administration may be considered when determining the therapeutically effective amount. In determining therapeutically effective amounts, one skilled in the art may also consider the existence, nature, and extent of any adverse effects that accompany the administration of a particular compound in a particular subject.

When used in the treatments described herein, a therapeutically effective amount of a composition can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to, for example, reduce cancer progression.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a delivery system composition can occur as a single event or over a time course of treatment. For example, a delivery system composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

In preferred aspects, a method of the disclosure is used to treat a neoplasm or cancer. The neoplasm may be malignant or benign, the cancer may be primary or metastatic; the neoplasm or cancer may be early stage or late stage. A cancer or a neoplasm may be treated by delivering delivery system of the disclosure labeled with a therapeutic agent to at least one cancer cell in a subject. The cancer or neoplasm may be treated by slowing cancer cell growth or killing cancer cells.

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

Non-limiting examples of neoplasms or cancers that may be treated with a method of the invention may include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenstrom), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-cell lymphoma (cutaneous), T-cell leukemia and lymphoma, testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, or Wilms tumor (childhood).

In other aspects, nanoparticles of the disclosure may be delivered to a cancer cell in vitro. A cancer cell may be a cancer cell line cultured in vitro. In some alternatives of the embodiments, a cancer cell line may be a primary cell line that is not yet described. Methods of preparing a primary cancer cell line utilize standard techniques known to individuals skilled in the art. In other alternatives, a cancer cell line may be an established cancer cell line. A cancer cell line may be adherent or non-adherent, or a cell line may be grown under conditions that encourage adherent, non-adherent or organotypic growth using standard techniques known to individuals skilled in the art. A cancer cell line may be contact inhibited or non-contact inhibited.

In some embodiments, the cancer cell line may be an established human cell line derived from a tumor. Non-limiting examples of cancer cell lines derived from a tumor may include the MM cell lines MM.1S, H929, and RPMI, osteosarcoma cell lines 143B, CAL-72, G-292, HOS, KHOS, MG-63, Saos-2, or U-2 OS; the prostate cancer cell lines DU145, PC3 or Lncap; the breast cancer cell lines MCF-7, MDA-MB-438 or T47D; the myeloid leukemia cell line THP-1, the glioblastoma cell line U87; the neuroblastoma cell line SHSY5Y; the bone cancer cell line Saos-2; the colon cancer cell lines WiDr, COLO 320DM, HT29, DLD-1, COLO 205, COLO 201, HCT-15, SW620, LoVo, SW403, SW403, SW1116, SW1463, SW837, SW948, SW1417, GPC-16, HCT-8, HCT 116, NCI-H716, NCI-H747, NCI-H508, NCI-H498, COLO 320HSR, SNU-C2A, LS 180, LS 174T, MOLT-4, LS513, LS1034, LS411N, Hs 675.T, CO 88BV59-1, Co88BV59H21-2, Co88BV59H21-2V67-66, 1116-NS-19-9, TA 99, AS 33, TS 106, Caco-2, HT-29, SK-CO-1, SNU-C2B or SW480; B16-F10, RAW264.7, the F8 cell line, or the pancreatic carcinoma cell line Panc1. In an exemplary embodiment, a method of the disclosure may be used to contact a cell of a MM cell line.

### III. Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to compositions and pharmaceutical formulations comprising a nanoparticle composition, as described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Specific embodiments disclosed herein may be further limited in the claims using “consisting of” or “consisting essentially of” language, rather than “comprising”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As various changes could be made in the above-described materials and methods without departing from the scope of the invention, it is intended that all matter contained in the above description and in the examples given below, shall be interpreted as illustrative and not in a limiting sense.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Nanoparticle Tracking Analysis and Generation of Reactive Oxygen Species

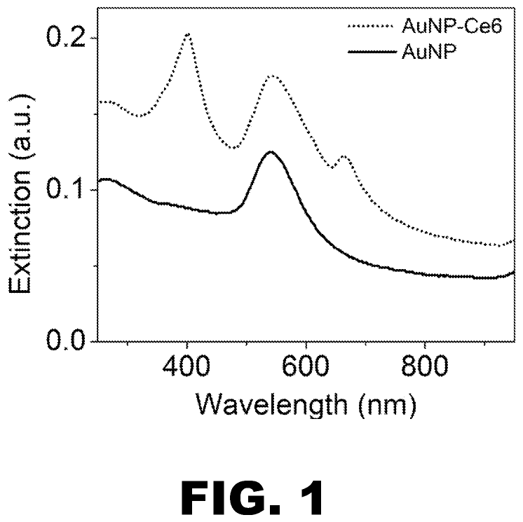

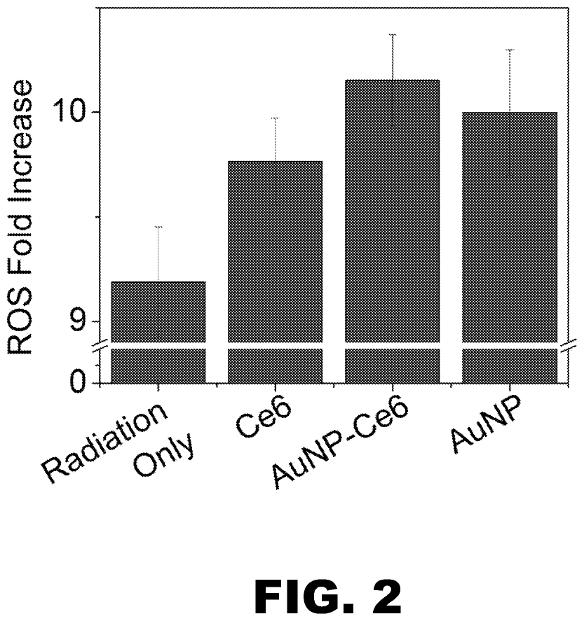

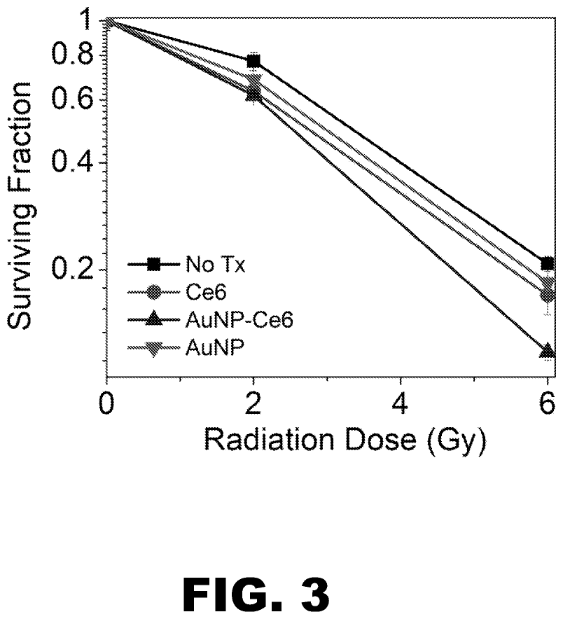

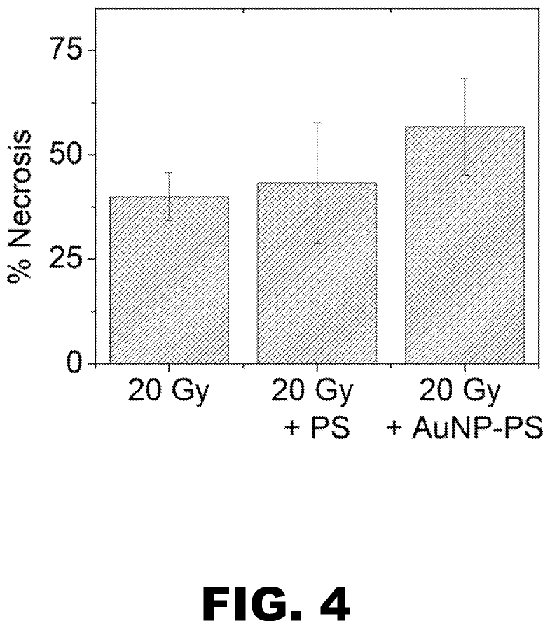

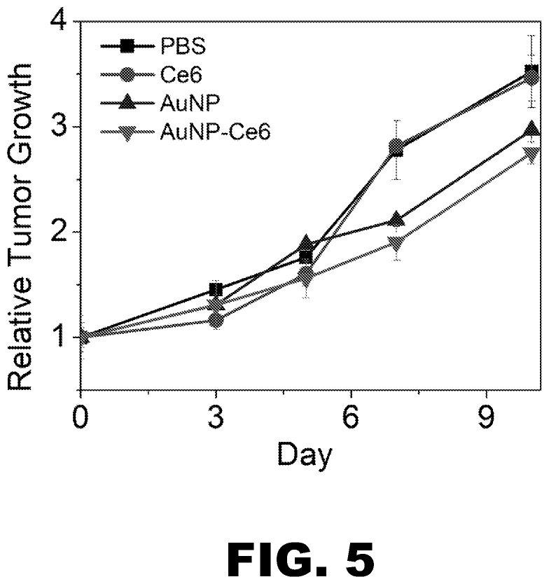

Amine terminated, PEGylated Au nanospheres were purchased from a commercial supplier, and conjugated with chlorin e6 using EDC/NHS chemistry in MES buffer at pH=5 overnight. The conjugated particles were collected via centrifugation and washed with methanol to remove unconjugated Ce6. The UV-Vis spectrum (FIG. 1) showed characteristic peaks for Ce6, and no appreciable shift in the peak from the gold nanoparticle. This lack of shift suggests the particles were not aggregated, which was confirmed using nanoparticle tracking analysis where the mean particle hydrodynamic diameter decreased from 100 nm to 80 nm following conjugation with Ce6. ROS generation (FIG. 2) was measured using fluorescein diacetate staining (a known method of quantification of ROS generation) following overnight incubation of breast cancer cells with Ce6, Au nanoparticles, or the conjugate (at the same nanoparticle concentration). Cells were incubated with fluorescein diacetate for 30 min exposed to 10 Gy radiation (150 kV) and measurements were taken immediately following radiation. Clonogenic survival (FIG. 3) was measured by thinly seeding cancer cells in a 6-well plate and incubating overnight. Radiation doses of 0, 2, or 6 Gy were applied and the cells were allowed to grow for roughly 7 days prior to fixation and crystal violet staining. Groups of cells were considered colonies when numbering greater than 50 cells. The 2 Gy and 6 Gy groups were normalized to 0 Gy for that treatment condition, and a significant decrease in colonies was observed for the photosensitizer conjugated particle group. Tumor necrosis (FIG. 4) was assessed in mice bearing hind limb tumors. Intravenous injection of PBS, photosensitizer, or the conjugate was performed followed by 20 Gy irradiation within 30 minutes of injection. Mice were sacrificed after 3 days and the tumors were harvested, sectioned, stained for H&E and scored histopathologically. In a separate cohort of mice, tumor growth (FIG. 5) was measured. Rear limb tumors were again used and mice were injected intratumorally with PBS, photosensitizer, nanoparticle, or the conjugate and then received 20 Gy irradiation. Mice were monitored for 10 days and tumor growth recorded regularly. Both the nanoparticle groups showed decreased tumor volume relative to control by the end of the study.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

It should also be understood that, unless clearly indicated to the contrary, in any methods claimed herein that include more than one step or act, the order of the steps or acts of the method is not necessarily limited to the order in which the steps or acts of the method are recited.

As used herein the term “alkyl” can refer to C1-20 inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C1-8 alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C1-8 straight-chain alkyls. In other embodiments, “alkyl” refers, in particular, to C1-8 branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. In some embodiments, there can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

What is claimed is:

1. A nanoparticle composition, comprising:
a scintillation nanoparticle having a coating layer and a photosensitizer conjugated to the coating layer, wherein the scintillation nanoparticle emits electromagnetic radiation having a first wavelength when irradiated with electromagnetic radiation having a second wavelength and wherein the photosensitizer absorbs electromagnetic radiation of said first wavelength.

2. The nanoparticle composition of claim 1, wherein absorption of the first wavelength by the photosensitizer can activate the photosensitizer to produce singlet oxygen for photodynamic therapy.

3. The nanoparticle composition of claim 1 or claim 2, wherein the scintillation nanoparticle is a high-Z metal nanoparticle.

4. The nanoparticle composition of claim 3, wherein the high-Z metal nanoparticle is selected from gold, platinum, gadolinium, silver, titanium, zinc, cerium, iron, thallium, and a metal oxide.

5. The nanoparticle composition of claim 4, wherein the nanoparticle is a gold nanoparticle.

6. The nanoparticle composition of any one of claims 1-5, wherein the coating layer comprises one or more polyethylene glycol (PEG) group.

7. The nanoparticle composition of claim 6, wherein the PEG group is covalently linked to the photosensitizer.

8. The nanoparticle composition of any one of claims 1-7, wherein the photosensitizer is cyanine, porphyrin, pyrrole, tetrapyrollic compounds, expanded pyrrolic macrocycles, flavins, organometallic species, or combinations thereof.

9. The nanoparticle composition of any one of claims 1-7, wherein the photosensitizer is selected from the group consisting of merocyanine, phthalocyanine, chloroaluminum phthalocyanine, sulfonated aluminum phthalocyanine, ring-substituted cationic phthalocyanine, sulfonated aluminum naphthalocyanine, naphthalocyanine, tetracyanoethylene adduct, crystal violet, azure P chloride, benzophenothiazinium, benzophenothiazinium chloride (EtNBS), phenothiazine, rose Bengal, toluidine blue, toluidine blue O (TBO), methylene blue (MB), new methylene blue N (NMMB), new methylene blue BB, new methylene blue FR, 1,9-dimethylmethylene blue chloride (DMMB), methylene green, methylene violet Bernthsen, methylene violet 3RAX, Nile blue, malachite green, Azure blue A, Azure blue B, Azure blue C, safranine O, neutral red, 5-ethylamino-9-diethylaminobenzo[a]phenothiazinium chloride, 5-ethylamino-9-diethylaminobenzo[a]phenoselenazinium chloride, thiopyronine, and thionine.

10. The nanoparticle composition of any of claims 1-7, wherein the photosensitizer is Chlorin e6 (Ce6).

11. The nanoparticle composition of any one of claims 1-10, wherein the nanoparticle further comprises a cell targeting moiety conjugated to the coating layer.

12. The nanoparticle composition of claim 11, wherein the cell targeting moiety is selected from a receptor, ligand, polynucleotide, peptide, polynucleotide binding agent, antigen, antibody, or combinations thereof.

13. The nanoparticle composition of claim 12, wherein the cell targeting moiety is recognized by a neoplasm or cancer cell.

14. A pharmaceutical composition comprising at least one pharmaceutically acceptable excipient and a nanoparticle composition according to anyone of claims 1-13.

15. The pharmaceutical composition of claim 14, wherein the pharmaceutical composition is formulated for intravenous (i.v.), intraperitoneal (i.p.), intramuscular (i.m.), intratumoral (i.t), intraarterial (i.a.), or topical administration.

16. A method for photodynamic therapy in a subject in need thereof, the method comprising:
providing a nanoparticle composition comprising a scintillation nanoparticle having a coating layer and a photosensitizer conjugated to the coating layer, wherein the scintillation nanoparticle emits electromagnetic radiation having a first wavelength when irradiated with electromagnetic radiation having a second wavelength, wherein the photosensitizer absorbs electromagnetic radiation of said first wavelength;
administering to the subject an effective amount of a composition comprising the nanoparticle composition; and
administering to the subject electromagnetic radiation having a second wavelength.

17. The method of claim 16, wherein the electromagnetic radiation source produces radiation selected from the group consisting of X-rays, alpha particles, beta-particles, neutrons, gamma rays, and combinations thereof.

18. The method of claim 16 or claim 17, wherein the nanoparticle composition and the electromagnetic radiation provide the subject a combination therapy where the therapeutic results are additive or synergistic compared to either electromagnetic radiation or photodynamic therapy alone.

19. The method according to any one of claims 16-18, wherein the subject has a neoplasm of cancer.

20. The method of claim 19, wherein the neoplasm or cancer is treated.

21. The method according to any one of claims 16-20, wherein absorption of the first wavelength by the photosensitizer can activate the photosensitizer to produce singlet oxygen for photodynamic therapy.

22. The method according to any one of claims 16-21, wherein the scintillation nanoparticle is a high-Z metal nanoparticle.

23. The method according to any one of claims 16-22, wherein the high-Z metal nanoparticle is selected from gold, platinum, gadolinium, silver, titanium, zinc, cerium, iron, thallium, and a metal oxide.

24. The method according to any one of claims 16-23, wherein the nanoparticle is a gold nanoparticle.

25. The method according to any one of claims 16-24, wherein the coating layer comprises one or more polyethylene glycol (PEG) group.

26. The method according to any one of claims 16-25, wherein the PEG group is covalently linked to the photosensitizer.

27. The method according to any one of claims 16-26, wherein the photosensitizer is cyanine, porphyrin, pyrrole, tetrapyrollic compounds, expanded pyrrolic macrocycles, flavins, organometallic species, or combinations thereof.

28. The method according to any one of claims 16-27, wherein the photosensitizer is selected from the group consisting of merocyanine, phthalocyanine, chloroaluminum phthalocyanine, sulfonated aluminum phthalocyanine, ring-substituted cationic phthalocyanine, sulfonated aluminum naphthalocyanine, naphthalocyanine, tetracyanoethylene adduct, crystal violet, azure P chloride, benzophenothiazinium, benzophenothiazinium chloride (EtNBS), phenothiazine, rose Bengal, toluidine blue, toluidine blue O (TBO), methylene blue (MB), new methylene blue N (NMMB), new methylene blue BB, new methylene blue FR, 1,9-dimethylmethylene blue chloride (DMMB), methylene green, methylene violet Bernthsen, methylene violet 3RAX, Nile blue, malachite green, Azure blue A, Azure blue B, Azure blue C, safranine O, neutral red, 5-ethylamino-9-diethylaminobenzo[a]phenothiazinium chloride, 5-ethylamino-9-diethylaminobenzo[a]phenoselenazinium chloride, thiopyronine, and thionine.

29. The method according to any one of claims 16-28, wherein the photosensitizer is Chlorin e6 (Ce6).

30. The method according to any one of claims 16-29, wherein the nanoparticle further comprises a cell targeting moiety conjugated to the coating layer.

31. The method according to any one of claims 16-30, wherein the cell targeting moiety is selected from a receptor, ligand, polynucleotide, peptide, polynucleotide binding agent, antigen, antibody, or combinations thereof.

32. The method according to any one of claims 16-31, wherein the cell targeting moiety is recognized by a neoplasm or cancer cell.

33. A method for treating a neoplasm or cancer in a subject in need thereof, the method comprising:
providing a nanoparticle composition comprising a scintillation nanoparticle having a coating layer and a photosensitizer conjugated to the coating layer, wherein the scintillation nanoparticle emits electromagnetic radiation having a first wavelength when irradiated with electromagnetic radiation having a second wavelength, wherein the photosensitizer absorbs electromagnetic radiation of said first wavelength;
administering to the subject an effective amount of a composition comprising the nanoparticle composition; and
administering to the subject electromagnetic radiation having a second wavelength.

34. The method of claim 33, wherein the electromagnetic radiation source produces radiation selected from the group consisting of X-rays, alpha particles, beta-particles, neutrons, gamma rays, and combinations thereof.

35. The method of claim 33 or claim 34, wherein the nanoparticle composition and the electromagnetic radiation provide the subject a combination therapy where the therapeutic results are additive or synergistic compared to either electromagnetic radiation or photodynamic therapy alone.

36. The method according to any one of claims 33-35, wherein the subject has a neoplasm of cancer.

37. The method of claim 36, wherein the neoplasm or cancer is treated.

38. The method according to any one of claims 33-37, wherein absorption of the first wavelength by the photosensitizer can activate the photosensitizer to produce singlet oxygen for photodynamic therapy.

39. The method according to any one of claims 33-38, wherein the scintillation nanoparticle is a high-Z metal nanoparticle.

40. The method according to any one of claims 33-39, wherein the high-Z metal nanoparticle is selected from gold, platinum, gadolinium, silver, titanium, zinc, cerium, iron, thallium, and a metal oxide.

41. The method according to any one of claims 33-40, wherein the nanoparticle is a gold nanoparticle.

42. The method according to any one of claims 33-41, wherein the coating layer comprises one or more polyethylene glycol (PEG) group.

43. The method according to any one of claims 33-42, wherein the PEG group is covalently linked to the photosensitizer.

44. The method according to any one of claims 33-43, wherein the photosensitizer is cyanine, porphyrin, pyrrole, tetrapyrollic compounds, expanded pyrrolic macrocycles, flavins, organometallic species, or combinations thereof.

45. The method according to any one of claims 33-44, wherein the photosensitizer is selected from the group consisting of merocyanine, phthalocyanine, chloroaluminum phthalocyanine, sulfonated aluminum phthalocyanine, ring-substituted cationic phthalocyanine, sulfonated aluminum naphthalocyanine, naphthalocyanine, tetracyanoethylene adduct, crystal violet, azure P chloride, benzophenothiazinium, benzophenothiazinium chloride (EtNBS), phenothiazine, rose Bengal, toluidine blue, toluidine blue O (TBO), methylene blue (MB), new methylene blue N (NMMB), new methylene blue BB, new methylene blue FR, 1,9-dimethylmethylene blue chloride (DMMB), methylene green, methylene violet Bernthsen, methylene violet 3RAX, Nile blue, malachite green, Azure blue A, Azure blue B, Azure blue C, safranine O, neutral red, 5-ethylamino-9-diethylaminobenzo[a]phenothiazinium chloride, 5-ethylamino-9-diethylaminobenzo[a]phenoselenazinium chloride, thiopyronine, and thionine.

46. The method according to any one of claims 33-45, wherein the photosensitizer is Chlorin e6 (Ce6).

47. The method according to any one of claims 33-46, wherein the nanoparticle further comprises a cell targeting moiety conjugated to the coating layer.

48. The method according to any one of claims 33-47, wherein the cell targeting moiety is selected from a receptor, ligand, polynucleotide, peptide, polynucleotide binding agent, antigen, antibody, or combinations thereof.

49. The method according to any one of claims 33-48 wherein the cell targeting moiety is recognized by a neoplasm or cancer cell.

50. A nanoparticle composition, comprising:
a scintillation nanoparticle and a photosensitizer conjugated to the surface of the nanoparticle, wherein the scintillation nanoparticle emits electromagnetic radiation having a first wavelength when irradiated with electromagnetic radiation having a second wavelength and wherein the photosensitizer absorbs electromagnetic radiation of said first wavelength.

51. The nanoparticle composition of claim 50, wherein absorption of the first wavelength by the photosensitizer can activate the photosensitizer to produce singlet oxygen for photodynamic therapy.

52. The nanoparticle composition of claim 50 or claim 51, wherein the scintillation nanoparticle is a high-Z metal nanoparticle.

53. The nanoparticle composition of claim 52, wherein the high-Z metal nanoparticle is selected from gold, platinum, gadolinium, silver, titanium, zinc, cerium, iron, thallium, and a metal oxide.

54. The nanoparticle composition of claim 53, wherein the nanoparticle is a gold nanoparticle.

55. The nanoparticle composition of any one of claims 50-54, wherein the photosensitizer is cyanine, porphyrin, pyrrole, tetrapyrollic compounds, expanded pyrrolic macrocycles, flavins, organometallic species, or combinations thereof.

56. The nanoparticle composition of any one of claims 50-54, wherein the photosensitizer is selected from the group consisting of merocyanine, phthalocyanine, chloroaluminum phthalocyanine, sulfonated aluminum phthalocyanine, ring-substituted cationic phthalocyanine, sulfonated aluminum naphthalocyanine, naphthalocyanine, tetracyanoethylene adduct, crystal violet, azure P chloride, benzophenothiazinium, benzophenothiazinium chloride (EtNBS), phenothiazine, rose Bengal, toluidine blue, toluidine blue O (TBO), methylene blue (MB), new methylene blue N (NMMB), new methylene blue BB, new methylene blue FR, 1,9-dimethylmethylene blue chloride (DMMB), methylene green, methylene violet Bernthsen, methylene violet 3RAX, Nile blue, malachite green, Azure blue A, Azure blue B, Azure blue C, safranine O, neutral red, 5-ethylamino-9-diethylaminobenzo[a]phenothiazinium chloride, 5-ethylamino-9-diethylaminobenzo[a]phenoselenazinium chloride, thiopyronine, and thionine.

57. The nanoparticle composition of any of claims 50-54, wherein the photosensitizer is Chlorin e6 (Ce6).

58. The nanoparticle composition of any one of claims 50-57, wherein the nanoparticle further comprises a cell targeting moiety.

59. The nanoparticle composition of claim 58, wherein the cell targeting moiety is selected from a receptor, ligand, polynucleotide, peptide, polynucleotide binding agent, antigen, antibody, or combinations thereof.

60. The nanoparticle composition of claim 58, wherein the cell targeting moiety is recognized by a neoplasm or cancer cell.

61. A pharmaceutical composition comprising at least one pharmaceutically acceptable excipient and a nanoparticle composition according to anyone of claims 50-60.
